(19) United States
(12) Patent Application Publication
(10) Pub. No.: US 2014/0371829 A1
(43) Pub. Date: Dec. 18, 2014

(54) METHODS AND APPARATUS FOR A THERAPEUTIC DEVICE

(71) Applicant: Ten Minute, Inc., Cedar City, UT (US)

(72) Inventors: George W. Stine, Cedar City, UT (US); Lisa K. Stine, Cedar City, UT (US)

(21) Appl. No.: 14/304,372

(22) Filed: Jun. 13, 2014

Related U.S. Application Data
(60) Provisional application No. 61/835,347, filed on Jun. 14, 2013.

(51) Int. Cl. A61F 7/10

(52) U.S. Cl. CPC A61F 7/10
USPC 607/108

(57) ABSTRACT

Apparatus and methods according to various aspects of the present technology may comprise a treatment device configured for localized application of temperature treatment to aid in the therapeutic treatment of hemorrhoid disease. In one embodiment, the treatment device may comprise a body suitably configured to be placed directly against an external portion of an intergluteal space proximate to an anal verge of a user to allow localized exchange of heat energy between the external portion of the intergluteal space and composition disposed within a cavity of the body. In one embodiment, the body may be configured to be retained in position against the treatment area during use and to decrease a possibility of being inadvertently displaced after being positioned for use.

### CROSS-REFERENCE TO RELATED APPLICATIONS

This application claims the benefit of U.S. Provisional Application Ser. No. 61/835,347, filed Jun. 14, 2013, and incorporates the disclosure of such application in its entirety by reference. To the extent that the present disclosure conflicts with the referenced application, however the present disclosure is to be given priority.

### BACKGROUND

Hemorrhoids are fibrovascular cushions lining the anal canal comprised of blood vessels, smooth muscle, elastic tissue and connective tissue. Hemorrhoids are located in the upper anal canal at three consistent anatomical sites of the anal canal: the left lateral, the right anterolateral, and the right posterolateral quadrant. Hemorrhoids aid in the control of fecal continence and evacuation.

“Hemorrhoid disease” presents when hemorrhoids become pathlogical, for example, varicose, distended, swollen, inflamed, irritated or otherwise symptomatic. Pathological hemorrhoids comprise two categories: internal hemorrhoids and external hemorrhoids. Internal hemorrhoids originate above the dentate line; external hemorrhoids originate below the dentate line.

Common treatment methods for pathological hemorrhoids include treatment options such as surgical intervention, ligation, sclerotherapy, cauterization, dietary changes, application of topical agents, and sitz baths. In addition, use of therapeutic devices may allow for localized application of cryotherapy, thermotherapy and/or a combination thereof to the human body to aid in the therapeutic treatment of hemorrhoid disease is common. However, current therapeutic devices designed for application to an external anatomic surface limit the mobility of a user. Further, current therapeutic devices are not configured to retain position and to decrease inadvertent expulsion upon disposition proximate to an anal verge of the user.

### BRIEF SUMMARY

Apparatus and methods according to various aspects of the present technology may comprise a treatment device configured for localized application of temperature treatment to aid in the therapeutic treatment of hemorrhoid disease. In one embodiment, the treatment device may comprise a body suitably configured to be placed directly against an external portion of an intergluteal space proximate to an anal verge of a user to allow localized exchange of heat energy between the external portion of the intergluteal space and a composition disposed within a cavity of the body. In one embodiment, the body may be configured to be retained in position against the treatment area during use and to decrease a possibility of being inadvertently displaced after being positioned for use.

### BRIEF DESCRIPTION OF THE DRAWINGS

A more complete understanding of the present technology may be derived by referring to the detailed description when considered in connection with the following illustrative figures. In the following figures, like reference numbers refer to similar elements and steps throughout the figures.

Elements and steps in the figures are illustrated for simplicity and clarity and have not necessarily been rendered according to any particular sequence or scale. For example, steps that may be performed concurrently or in different order are illustrated in the figures to help to improve understanding of embodiments of the present technology.

The figures described are for illustration purposes only and are not intended to limit the scope of the present disclosure in any way. Various aspects of the present technology may be more fully understood from the detailed description and the accompanying drawing figures, wherein:

FIG. 1A representatively illustrates a therapeutic device in accordance with an exemplary embodiment of the present technology;

FIG. 1B representatively illustrates an end view of the therapeutic device in accordance with an exemplary embodiment of the present technology;

FIG. 1C representatively illustrates a perspective view of the therapeutic device in accordance with an exemplary embodiment of the present technology,

FIG. 2A representatively illustrates a posterior view of human buttocks showing the therapeutic device disposed at an external portion of the intergluteal space proximate to the anal verge;

FIG. 2B representatively illustrates a median sagittal section of human buttocks showing the therapeutic device disposed at an external portion of the intergluteal space proximate to the anal verge;

FIG. 3 representatively illustrates a cross-sectional view of the therapeutic device across line A-A as shown in FIG. 1A in accordance with an exemplary embodiment of the present technology; and

FIG. 4 representatively illustrates a cover for the therapeutic device in accordance with an exemplary embodiment of the present technology.

### DETAILED DESCRIPTION OF EXEMPLARY EMBODIMENTS

The present teleology may be described in terms of functional block components and various processing steps. Such functional blocks may be realized by any number of components configured to perform the specified functions and achieve the various results. For example, methods and systems according to various aspects of the present technology may be practiced in conjunction with any number of methods for localized application of temperature therapy to the human body to aid in therapeutic treatment by heating or cooling of a desired portion of the human anatomy and the system described is merely one exemplary application for the technology. Various representative implementations of the present technology may be applied to any type of conventional therapeutic device for localized application of temperature therapy to the human body.

The particular implementations shown and described are illustrative of the technology and its best mode and are not intended to otherwise limit the scope of the present technology in any way. For the sake of brevity, conventional manufacturing, connection, preparation, and other functional aspects of the system may not be described in detail. Furthermore, the connecting lines shown in the various figures are intended to represent exemplary functional relationships and/or steps between the various elements. Many alternative or additional functional relationships or physical connections may he present in a practical system.

Various aspects of the technology provide methods and apparatus for making and using a therapeutic device for localized application of temperature therapy to the human body to aid in the therapeutic treatment by heating or cooling as desired portion of the human anatomy according to a treatment plan. In one embodiment, the therapeutic device may be adapted for localized application of temperature therapy to the human body to aid in the treatment: of varicose, distended, swollen, inflamed, irritated, or otherwise symptomatic hemorrhoids, or hemorrhoid disease. Various representative implementations of the present technology may be applied to any appropriate system for therapeutic treatment of hemorrhoid disease. Certain representative implementations may include, for example, providing any suitable system, apparatus and method for applying temperature therapy, such as cryotherapy, thermotherapy, and/or a combination of cryotherapy and thermotherapy, to an external anatomical surface of an intergluteal space proximate an anal verge of a user to aid in therapeutic treatment of hemorrhoid disease.

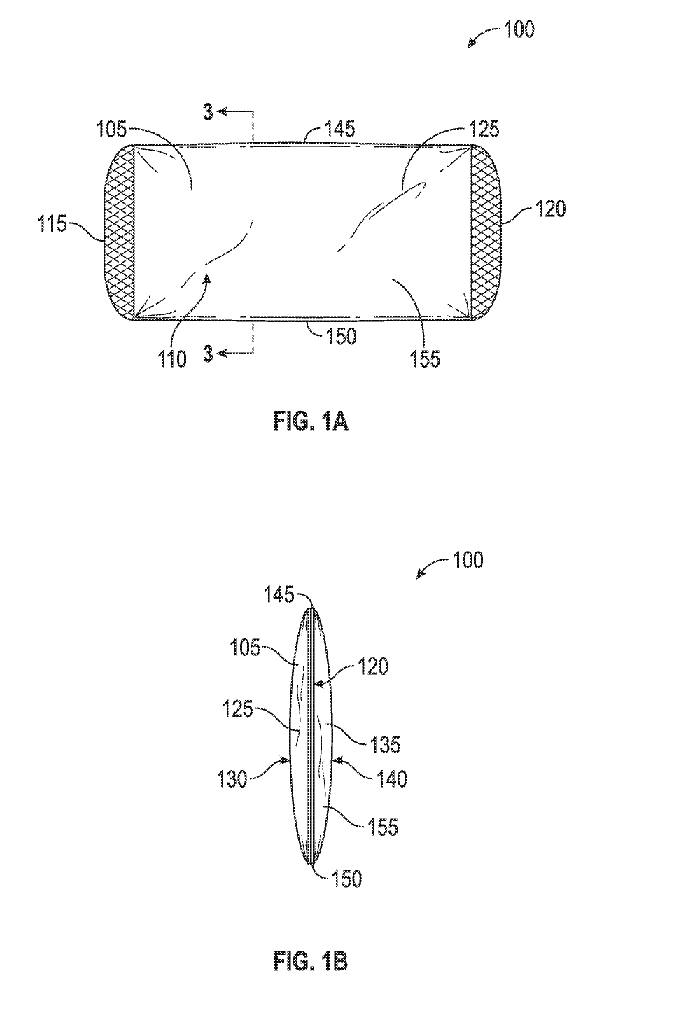

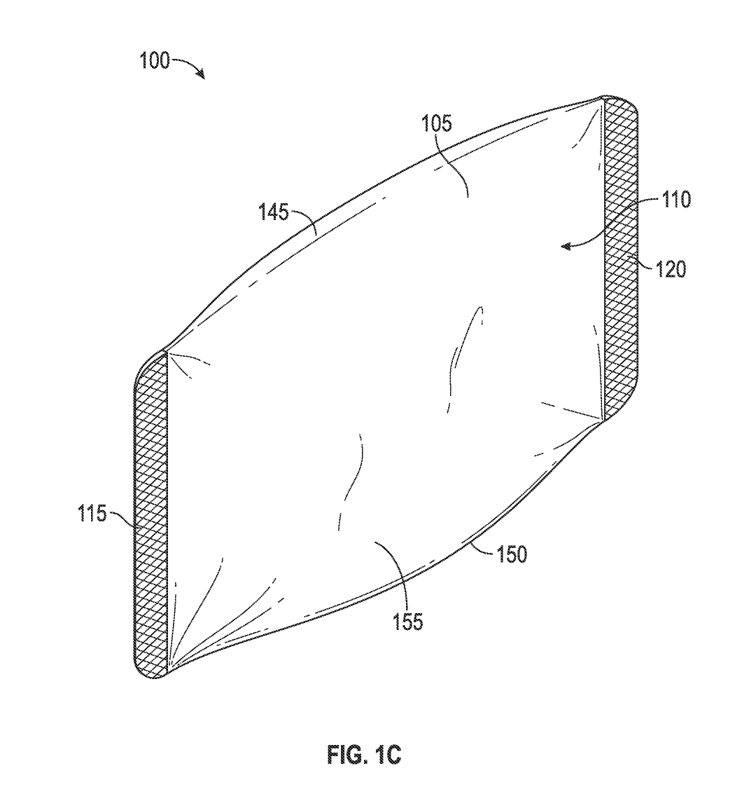

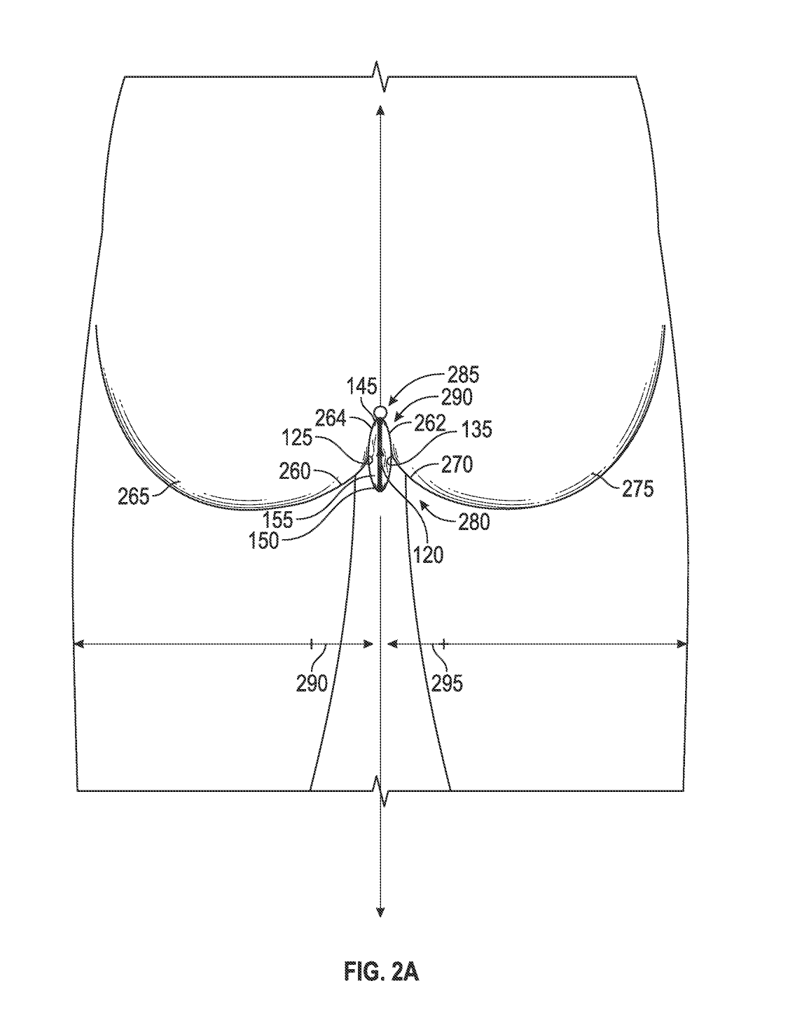

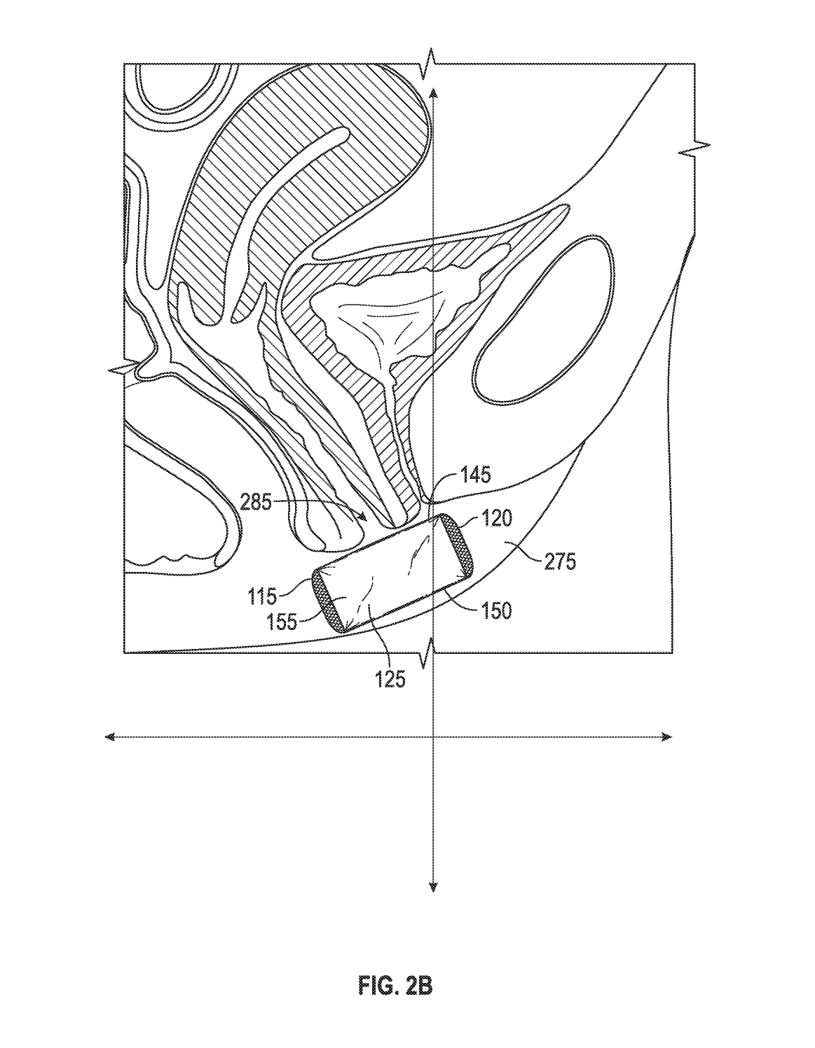

Referring to FIGS. 1A-1C and 2A-B, the therapeutic device 100 may comprise a body 105 having a cavity 110 and a composition 155 disposed within the cavity 110. The body 105 may be suitably configured to provide localized application of temperature therapy to the human body and may comprise any suitable device or system adapted for placement proximate, adjacent, or against a treatment area such as an external portion of an intergluteal space 280 of a user to allow for the localized exchange of heat energy between the treatment area and the composition 155 disposed within the cavity 110 of the body 105.

In one embodiment, the body 105 may comprise at pair of opposing pressure surfaces 125, 135 each extending between a first end portion 115 and a second end portion 120 of the body 105. The pair of opposing pressure surfaces 125. 135 may be coupled together along their ends to at least partially define the first and second end portions 115, 120. The pair of opposing pressure surfaces 125, 135 may be coupled together by any suitable method such as adhesively, plastic welding, heat fusion, or the like.

The body 105 may further comprise a pair of opposing treatment surfaces 145. 150 disposed between the pair of opposing pressure surfaces 125, 135 and extending between the first and second end portions 115, 120. The pair of opposing treatment surfaces 145, 150 may be configured to engage the pair of opposing pressure surfaces 125, 135 to define the cavity 110 as an inner volume of the body 105 separating the pair of opposing pressure surfaces 125, 135 and the pair of opposing treatment surfaces 145, 150 from each other.

The body 105 may comprise any suitable dimensions adapted to allow the body 105 to easily fit against, confirm to, or otherwise contour to a particular anatomical location such as the treatment area where localized application of temperature therapy is desired. For example, the body 105 may comprise a size and shape such as a substantially tubular structure, a packet-shaped structure, a box-shaped structure or the like that is suitably configured for placement at any external surface of the intergluteal space of the user. In one embodiment, the body 105 may comprise an overall length of between two to five inches, a width of between one-half of an inch to three inches, and a thickness of between one-quarter of an inch to three inches. In a second embodiment, the body 105 may comprise dimensions of approximately three inches by one inch by three-eighths of an inch. In yet another embodiment, the body 105 may be sized to correspond to the dimensions of the external portion of the intergluteal space 280 of a particular user.

In various embodiments, any one or more of the first end portion 115, the second portion 120, the pair of opposing pressure surfaces 125, 135 and the pair of opposing treatment surfaces 145, 150 may be suitably contoured and/or curved in any suitable manner to reduce any sharp edges, protrusions, or the like along external surfaces that might be uncomfortable to the user during use. For example, referring now to FIGS. 1B and 2A, in one embodiment, the pair of opposing treatment surfaces 145, 150 may comprise a pair of continuous, substantially rounded edges having a generally convex shape contoured to conformably engage the external portion of the intergluteal space 280 of the anal verge 285 of the user. The pair of opposing pressure surfaces 125, 135 may comprise a pair of continuous surfaces configured to contour to the external portion of the intergluteal space 280 between the user's laterally adjacent buttocks 265, 275 in response to a pressure applied by the buttocks 265, 275. The first and second end portions 115, 120 may also he contoured and/or curved in any suitable manner to reduce any external surfaces that might be uncomfortable during use. For example, the first and second end portions 115, 120 may be contoured to reduce friction, rubbing, abrasion, sharp edges, or other irritation during use.

The cavity 110 may contain a predetermined volume of the composition 155 and may comprise any suitable dimensions adapted store to desired volume of the composition 155. The composition 155 may comprise any suitable material configured to accumulate, generate, retain, transfer and/or exchange heat energy. The cavity 110 may further comprise one or more zones (not shown) and one or more separators (not shown) to provide communication between the zones. In one embodiment, the cavity 110 may be sealed, for example to maintain the integrity of the composition 155 upon exposure to various temperature ranges, to prevent loss of the composition 155 to the external environment, and/or to maintain a particular environment, shape and/or size within the cavity 110. In a second embodiment, the cavity may be selectively accessible to allow for the addition or removal of the composition 155.

The composition 155 provides a source of therapeutic treatment for the body 105. The composition 155 may comprise any suitable material, composition, or agent for providing therapeutic thermal treatment to a portion of the human anatomy. For example, the composition 155 may comprise a liquid, semi-liquid, gel, or paste adapted to provide hot and/or cold therapy over a period of time. The composition 155 may further be configured to be non-toxic to the skin to reduce a likelihood of injury in the event that the composition 155 comes into direct contact with the user's skin.

The composition 155 may comprise activators initiating exothermic and/or endothermic chemical reactions, air-activated reactions, or any other mobile thermoreaction or cryoreaction to generate a desired therapeutic temperature. The composition 155 may be adapted to retain a therapeutic temperature state for a prescribed treatment period when exposed to direct contact with the user's skin. For example. the composition 155 may comprise a non-toxic gel suitably adapted to retain a semi-solid state over a range of temperature values. In one embodiment, the composition 155 may comprise a material adapted to maintain a gel-like consistency when cooled to a range of about −12 degrees Celsius to about 7 degrees Celsius (about 10° F. to about 45° F.), and once at a desired temperature, provide cooling relief to the skin over a predetermined period of about five to about fifteen minutes. In another embodiment, the composition 155 may comprise a composition adapted to provide cooling relief for up to about 30 minutes when applied to the skin. In yet another embodiment, the composition 155 may comprise a material adapted to maintain a gel-like consistency when heated to a range of about 32° C. to about 0° C. (about 90° F. to about 140° F.), and once at a desired temperature, provide heating relief to the skin over a period of about live to about fifteen minutes.

The composition 155 may comprise any composition such that the body 105 containing the composition 155 may be suitably configured to be adaptable to easily fit against, conform to, or otherwise contour to a particular anatomical location. In one embodiment, the composition 155 may comprise a gel having a composition adapted to prevent solid freezing of the gel when exposed to temperatures associated with common residential refrigerators and/or freezers, for example, the gel may be adapted to remain a non-solid when exposed to temperature range of and obtaining an internal temperature of about −17° C. to about 2° C. (about 0° F. to about 35° F.) such that the body 105 is at least partially deformable and/or capable of at least partially conforming to the user's anatomy near the anal canal.

The composition may comprise any suitable non-toxic and/or non-caustic compound or substance such as thickening agents, freezing point depressents, emulsifiers, stabilizers, solubilizers, neutralizers, buffers, alcohols, solvents, and/or acidifiers. In some embodiments, the composition may comprise a compound or substance that may provide an aesthetically desirable property such as a water-soluble fragrance, dye, and/or pigment. For example, in one embodiment, the composition 155 may comprise a food-grade formulation of up to about 85% D-glucitol, up to about 25% water, up to about 3% of a cross-linked polyacrylate copolymer, up to about 2% coloring, and up to about 2% methylchloroisotinazolinone,

The composition 155 may be reusable or disposable. For example, one or more compositions 155 may be removably disposed within the cavity 110 and/or the one or more zones (not shown). For example, the composition 155 may comprise a self-contained, removable gel packet, In one embodiment, the composition 155 may comprise a disposable unit that is disposed after one use.

Referring now to FIGS. 1A-1C and 2A-2B, the pair of opposing pressure surfaces 125, 135 may he adapted to conformably engage the external portion of the intergluteal space 280 inferior to the anal verge 285, such as external skin surfaces of a user's laterally adjacent buttocks 265, 275 comprising an external skin surface portion 260 of the first buttock 265 and a second external surface area 270 of the opposing second buttock 275. For example, in one embodiment. the opposing pressure surfaces 125, 130 may comprise a first external surface area 135 and a second external surface area 140. The first external surface area 135 may be configured to engage the external skin surface portion 260 of the first buttock 265 such that substantially all of the first external surface area 135 is in contact with the first buttock 265. Similarly, the second pressure surface area. 140 may be configured to engage the external skin surface portion 270 of the opposing second buttock 275 such that substantially all of the second external surface area 270 is in contact with the opposing second buttock 275.

The pair of opposing treatment surfaces 145, 150 may be adapted to conformably engage an external portion of the intergluteal space 280 proximate to the anal verge 285, such as external skin surfaces disposed at an intergluteal fold 290 comprising a third external skin surface portion 262 of the first buttock 265 and a fourth external surface area 264 of the opposing second buttock 275. During use, at least one of the pair of opposing treatment suffices 145, 150 may engage the third and fourth external skin surface portions 262, 264 such that substantially all of the at least one of the pair of opposing treatment surface 145, 150 is in contact with a area at least immediately proximate to the anal verge 285.

With continuing to reference FIGS. 1A-1C and 2A-2B, the 105 of the therapeutic device 100 may be configured to be retained in position at the external portion of the intergluteal space 280 upon disposition for application of temperature therapy proximate to in anal verge 285 of the user. In one embodiment, the body 105 of the therapeutic device 100 may be suitably configured to maintain a pre-set position and to decrease inadvertent expulsion upon disposition at the external portion of the intergluteal space 280 of the user by conformably engaging the user's laterally adjacent buttocks 265, 275. For example, the first pressure surface 125 and the second pressure surface 130 may be configured to conformably engage the user's laterally adjacent buttocks 265, 275 and deform in response to a medial compression force 290, 295 applied by the user's laterally adjacent buttocks 265, 275, wherein the medial compression force 290, 295 is defined as a degree of force applied by laterally adjacent buttocks to the device at their points of contact with the pair of opposing pressure surfaces 125, 135. In one embodiment, the medial compression force 290, 295 applied by the user's laterally adjacent buttocks 265, 275 may cause the pair of opposing pressure surfaces 135, 135 to medially compress to cause at least one of the pair of opposing treatment surfaces 145, 150 to distend superiorly towards the anal verge 285 to engage the external skin surface portions 262, 264 at the external portion of the intergluteal space 280 proximate to the anal verge 285.

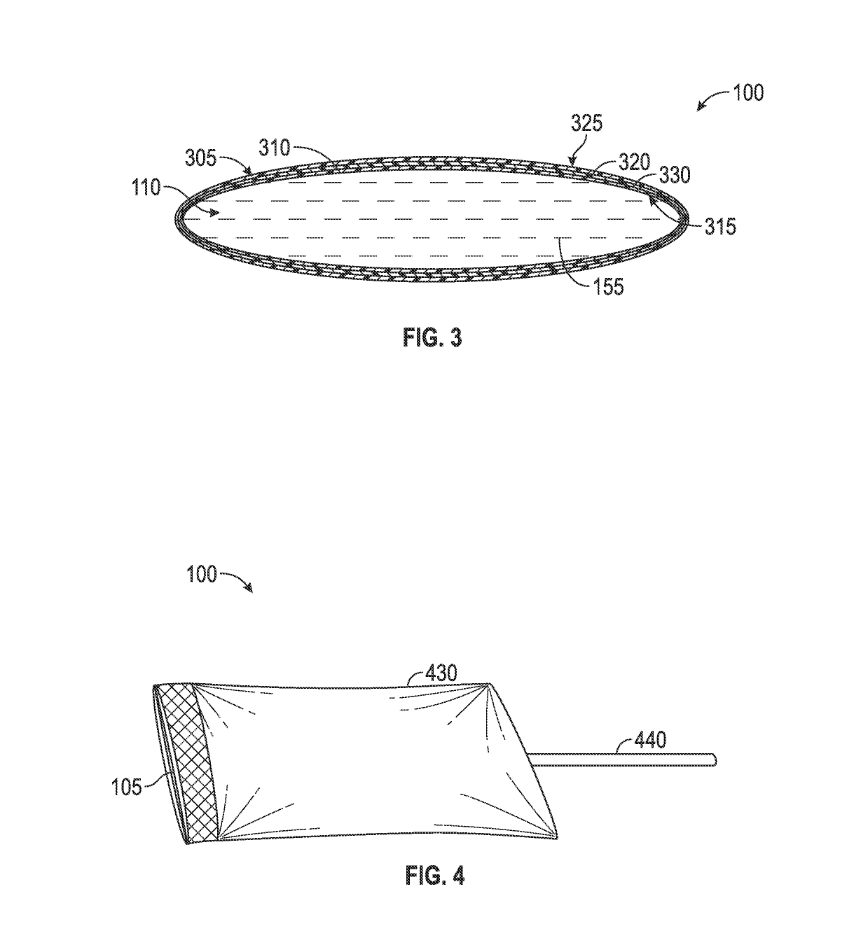

The body 105 may comprise any suitable structure adapted to allow exchange of heat energy between the external portion of the intergluteal space 280 and the composition 155 disposed within the cavity 110. Referring now to FIGS. 1A-1C, and 3, each of the pair of opposing pressure surfaces 125, 135 of the body 105 may comprise a plastic material having a three layer structure 305. In one embodiment, the three layer structure 305 may comprise an inner layer 310 comprising an inner surface area 315 defining the cavity 110, an outer layer 320 comprising an outer surface area 325 of the body 105 distal to the cavity 110 and defining the exterior surfaces of the pair of opposing pressure surfaces 125, 135 and the pair of opposing treatment surfaces 140, 145, and an intermediate layer 330 disposed between the inner layer 310 and the outer layer 320.

The body 105 may be suitably configured to contribute to a pre-determined rate of thermal heat transfer between the therapeutic device 100 and the human body during use. For example, the three layer structure 305 of the body 105 may comprise a combination of materials and/or varying layer thicknesses to at least partially control the rate of thermal heat transfer between the composition 155 and the user and/or provide any desired properties such as a desired level of flexibility, durability, comfortability, responsiveness to an applied pressure, and the like to the body 105 during use. The rate of heat transfer may be controlled for any desired reason such as to prevent a user from being burned when the body 105 is placed directly against the skin or to provide a sufficient rate of thermal transfer to provide therapeutic benefits.

The three layer structure 305 of the body 105 may comprise any suitable uniform or variable thickness and may be adapted to facilitate, an exchange of heat energy between the external portion of the intergluteal space and the composition 155 disposed within the cavity 110. For example, in one embodiment, a collective thickness of the three layers may be about 0.13 mm (about 0.005 in) to about 0.42 mm (about 0.016 in) thick. In another embodiment, the collective thickness of the three layers may range from about 0.35 mm (about 0.014 in) to about 0.2 mm (about 0.015 in) thick.

The three layer structure 305 may be suitably adapted to reduce permeability of the body 105 to the composition 155. In one embodiment, the three layer structure 305 may he adapted to be substantially impermeable to the composition 120 disposed within the cavity 110. For example, one or more of the inner layer 310, intermediate layer 330 and/or the outer layer 320 may be configured to be substantially impermeable to the composition 120 disposed within the cavity 110. In one embodiment, the inner layer 310 may comprise a material thickness adapted to decrease permeability at an interface of the composition 155 disposed within the cavity 110 and the inner surface area 315 of the inner layer 310. In a second embodiment, the inner layer 310 may comprise a material configured to be substantially impermeable to the composition 120 disposed within the cavity 110.

The intermediate layer 330 may further he adapted to be imprinted with an ink or dye. For example, in one embodiment an exterior facing surface the intermediate layer 330 may be printed with a logo and/or instructions for use. A clear outer layer 320 may then he positioned over the intermediate layer 330 so that the ink does not come into direct contact with the user's skin. The outer layer 320 may also be configured to provide any desired material properties such as being: biocompatible, nonbioabsorbable, and/or non-immunogenic when positioned in direct contact at a desired anatomical location. The outer layer 320 may further be configured to provide reinforcement and/or improved mechanical integrity to the body 105.

The body 105 may comprise any suitable material adapted to provide any desired result or feature such as to facilitate and/or control a rate of heat transfer between the external portion of the intergluteal space and the composition 155, provide structural mechanical integrity to the body 105, antibacterial and/or antimicrobial properties, and the like. In some embodiments, the body 105 may comprise any natural or synthetic material that is biologically compatible with soft tissue and insoluble in body fluids. For example, in some embodiments, the body 105 may comprise a natural or synthetic polymer, ceramic, fabric, and/or a combination thereof. For example, the natural or synthetic polymer may comprise polymers such poly(ethylene terephthalate) (PET), polyethene (PE), polypropylene (PP), nylon (NY), polytetrafluorothylene (PTFE), polyvinyl chloride (PVC) polyvinylidene fluoride (PVDF) and/or any combination thereof.

The material of each of the inner layer 310, the intermediate layer 330, and the outer layer 320 of the three layer structure 305 may constitute a combination of polymers suitably configured for any desired purpose. In one embodiment, the three layer structure 305 of the body 105 may comprise a combination of FDA-grade PET, PE and/or NY. For example, referring again to FIG. 3, in one embodiment, the inner layer 310 may comprise NY having a thickness of about 0.14 mm (about 0.005 in), the intermediate layer 330 may comprise PE having a thickness of about 0.12 mm (about 0.004 in), and the outer layer 320 may comprise PET having a thickness of about 0.12 mm (about 0.004 in). In a second embodiment, the inner layer 310 may comprise PET having a thickness of between about 0.09 mm to about 0.14 mm (about 0.003 in to about 0.005 in), the intermediate layer 330 may comprise NY having a thickness of between about 0.09 mm to about 0.17 mm (about 0.003 in to about 0.007 in), and the outer layer 320 may comprise a PE having a thickness of between about 0.09 to about 0.14 mm (about 0.003 to about 0.005 in).

The outer surface area 325 of the body 105 may comprise a smooth surface, a surface texture, or a combination thereof. In one embodiment, the external surface area 325 of the outer layer 320 may comprise a surface texture (not shown) adapted to promote temporary adherence of the pair of opposing pressure surfaces 125, 135 of the body 105 to surrounding tissue, such as the external portion of the intergluteal space 280. In some embodiments, the surface texture may comprise raised microscale nodules, surface depressions or microscale depressions. The surface texture may comprise any suitable texture configured to promote the opposing pressure surfaces 125, 135 to retain the body 105 in a desired position against a user's laterally adjacent buttocks.

The body 105 may further he suitably adapted to be exposed to any suitable temperature range required to achieve a therapeutic temperature state of the composition 155 and/or to be effectively cleaned while still retaining its shape, size and therapeutic function. For example, the body 105 may be adapted for exposure to a temperature range between about 0° C. to about 100° C. (about 32° F. to about 212° F.). In one embodiment, the body 105 may comprise any suitable material resistant to repeated cooling and/or heating by water bath. In a second embodiment, the body 105 may comprise any suitable material resistant exothermic and/or endothermic chemical reactions, air-activated reactions, or any other mobile thermoreaction or cryoreaction.

The therapeutic device 100 may be reusable or disposable. For example, the outer layer 320 of the body 105 may be adaptable such that the outer layer 320 may be disinfected or otherwise cleaned between uses. In one embodiment, the body 105 may be adapted to be cleaned by various methods such as autoclaving, chemical application, irradiation, steam, water bath, and/or by soap and water.

Referring now to FIG. 4, the therapeutic device 100 may further comprise a cover 430 and/or a removal device 440 coupled to the body 105 or cover 430 to facilitate removal and/or manipulation of the therapeutic device 100. The cover 430 may comprise any suitable material adapted to transfer heat to comfortably provide therapeutic treatment while preventing thermal skin injury. For example, the material may comprise any suitable material such as a natural polymer, synthetic polymer, plastic, fabric, tissue, and the like. In one embodiment, the cover 430 may comprise a fabric material configured to be positioned over the body 105 prior to being applied to the skin to increase, decrease, stabilize, or otherwise help control heat transfer. The cover 430 may be sealed or resealed by any suitable method such as a button, zipper, and the like.

The cover 430 may be reusable or disposable. For example, in one embodiment, the cover 430 may be configured to be disinfected and/or otherwise cleaned between uses. In a second embodiment, the cover 430 may comprise a disposable material such as tissue paper that is wrapped around the body 105 during use and subsequently discarded.

The removal device 440 may allow the user to more effectively retrieve the body 105 following use. For example, the removal device 440 may comprise a handle, a string, and the like. The removal device 440 may be removably or irremovably coupled to the therapeutic device 100 or the cover 430.

The therapeutic device 100 may comprise one or more sensors and a feedback mechanism to indicate various conditions of the therapeutic device 100. The sensors may comprise any suitable system or device such as a temperature sensor, use sensor, time sensor, and the like. The feedback mechanism may comprise any human perceivable form to communicate a condition of the device such as color. In one embodiment, the sensor may comprise any suitable thermochromic indicator material that changes color in tandem with a change in temperature state. For example, the thermochromic indicator material may comprise any suitable material, such as a natural polymer, a synthetic polymer, plastic, and the like capable of indicating a plurality of color indicators such as a plurality of polythiophenes. Referring now to FIG. 3 and FIG. 4, the thermochromic indicator material may comprise a plurality of polythiophenes dispersed in the outer layer 320 of the body 105 or an outer layer of the cover 430 wherein each polytheiophene corresponds to a predetermined color which further corresponds to a predetermined, temperature range. In one embodiment, a blue color may be exhibited when the composition 155 disposed within the cavity 110 of the body 105 is in a semi-solid state. For example, the predetermined temperature range may comprise between about −12° C. to about 2° C. (about 10° F. to about 35° F.).

The therapeutic device 100 may also be configured for long or .short-term storage stability. For example, the composition 155 may comprise a room- temperature stable composition. The therapeutic device 100 may further comprise any suitable materials adaptable absorb, repel, or otherwise reduce odor absorption generated by the human. anatomy such as the anal canal. For example, the outer layer 325 may comprise any suitable material adapted to absorb, repel, or otherwise reduce odor absorption. In addition, the therapeutic device 100 may comprise any suitable material configured to provide moisture control. For example, the outer layer 325 may comprise any suitable material adapted to absorb, repel, or otherwise reduce moisture absorption.

In operation, a user may heat or cool the therapeutic device 100 according to a

desired therapeutic treatment plan. For example, if the user is desirous of using cold therapy to treat hemorrhoid disease, the user may first place the therapeutic device 100 into a standard household refrigerator for a period of up to an hour to reduce a temperature of a composition 155 inside the therapeutic device 100. Once a desired temperature level has been achieved, the user may position the therapeutic device 100 directly against an external portion of an intergluteal space proximate to an anal verge for a predetermined, period of between 5 and 30 minutes.

If the user is desirous of obtaining an enhanced cooling effect, the therapeutic device 100 may be placed into a standard household freezer to further reduce the temperature of the composition 155. To avoid damaging the skin, the user may opt to position the therapeutic device 100 into a cover 430 or wrap the therapeutic device 100 in a layer or two of tissue to prevent the therapeutic device 100 from coming into direct contact with the skin. The user may position the therapeutic device 100 directly against the external portion of the intergluteal space proximate to the anal verge for a period of between 5 and 30 minutes.

If the user is desirous of applying a heating therapy, a similar process could be used to heat the therapeutic device 100. For example, the therapeutic device 100 may be placed into a water bath for a few minutes to increase the temperature of the composition 155 to a desired treatment level. Thereafter, the user may position the therapeutic device 100 directly against an external portion of an intergluteal space proximate to an anal verge for a period of between about 5 minutes and about 30 minutes.

The particular implementations shown and described are illustrative of the technology and its best mode and are not intended to otherwise limit the scope of the present technology in any way. Indeed, for the sake of brevity, conventional manufacturing, connection, preparation, and other functional aspects of the system may not he described in detail. Furthermore, the connecting lines shown in the various figures are intended to represent exemplary functional relationships and/or steps between the various elements. Many alternative or additional functional relationships or physical connections may be present in a practical system.

In the foregoing description, the technology has been described with reference to specific exemplary embodiments. Various modifications and changes may be made, however, without departing from the scope of the present technology as set forth. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in an method or process embodiment may be executed in any appropriate order and are nut limited to the explicit order presented in the specific examples. Additionally, the components and/or elements recited in any system embodiment may be combined in a variety of permutations to produce substantially the same result as the present technology and are accordingly not limited to the specific configuration recited in the specific examples.

Benefits, other advantages and solutions to problems have been described above with regard to particular embodiments. Any benefit, advantage, solution to problems or any element that may cause any particular benefit, advantage or solution to occur or to become more pronounced, however, is not to be construed as a critical, required or essential feature or component.

The terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology.

What is claimed. is:

1. A therapeutic device for application to an external portion of an intergluteal space proximate an anal verge of a user:
a body having a cavity defined by:
a first end portion;
a second end portion;
a pair of opposing pressure surfaces extending between the first end portion and the second end portion comprising:
a first pressure surface having a first external surface area; and
a second pressure surface having a second external surface area;
a pair of opposing treatment surfaces each extending between the first end portion and the second end portion and engaging the pair of opposing pressure surfaces, wherein:
the first external surface area of the first pressure surface is configured to engage an external skin surface portion of a first buttock such that substantially all of the first external surface area of the first pressure surface is in contact with the first buttock;
the second external surface area of the second pressure surface is configured to engage an external skin surface portion of an opposing second buttock. such that substantially all of the second external surface area of the second pressure surface is in contact with the opposing second buttock; and
the first and second pressure surfaces are configured to:
retain the body in position against the first buttock and the opposing second buttock; and
cause at least one of the pair of opposing treatment surfaces to engage an external skin surface at the external portion of the intergluteal space proximate to the anal verge; and
a composition disposed within the cavity,

2. The therapeutic device of claim 1, wherein the body comprises a three layer structure comprising:
an inner layer comprising an inner surface area of the body and defining the cavity;
an outer layer comprising an outer surface area of the body distal to the cavity and defining the pair of opposing pressure surfaces and the pair of opposing treatment surfaces; and
an intermediate layer disposed between the inner layer and the outer layer.

3. The therapeutic device of claim 2, wherein each of the inner layer, the intermediate layer, and the outer layer comprise a thermoplastic.

4. The therapeutic device of claim 3, wherein each of the inner layer, the intermediate layer and the outer layer comprise a collective thickness adapted to allow exchange of heat energy between the external portion of the intergluteal space and the composition.

5. The therapeutic device of claim 2, wherein the inner layer is adapted to be substantially impermeable to the composition disposed within the cavity.

6. The therapeutic device of claim 1, wherein the pair of opposing pressure surfaces and the pair of opposing treatment surfaces are contoured to the shape and size of the external portion of an intergluteal space.

7. The therapeutic device of claim 1, wherein the composition is adapted to remain a non-solid when exposed to a temperature range of about −12 degrees Celsius to about 2 degrees Celsius.

8. The therapeutic device of claim 1, wherein the composition comprises a gel.

9. The therapeutic device of claim 8, wherein the gel comprises:
up to about 85% D-glucitol;
up to about 3% of a cross-linked polyacrylate copolymer; and
up to about 2% methylchloroisothiazolinone

10. The therapeutic device of claim 9, wherein the gel further comprises:
up to about 25% water; and
up to about 2% coloring.

11. The therapeutic device of claim 8, wherein the gel is non-toxic to living tissue.

12. The therapeutic device of claim 1, wherein the therapeutic device further comprises a cover adapted to removably enclose at least a portion of the body.

13. A therapeutic device fir application to an external portion of an intergluteal space proximate an anal verge of a user:
a first end portion;
a second end portion;
a first pressure surface having a first external surface area extending between the first end portion and the second end portion;
a second pressure surface having a second external surface area extending between the first end portion and the second end portion and substantially opposing the first pressure surface;
a pair of opposing treatment surfaces each extending between the first end portion and the second end portion and engaging the pair of opposing pressure surfaces to define an interior cavity, wherein:
the first external surface area of the first pressure surface is configured to engage an external skin surface portion of a first buttock such that substantially all of the first external surface area of the first pressure surface is in contact with the first buttock;
the second external surface area of the second pressure surface is configured to engage an external skin surface portion of an opposing second buttock such that substantially all of the second external surface area of the second pressure surface is in contact with the opposing second buttock; and
the first and second pressure surfaces are configured to cause at least one of the pair of opposing treatment surfaces to engage an external skin surface at the external portion of the intergluteal space proximate to the anal verge; and
a composition on disposed with in the cavity.

14. The therapeutic device of claim 13, wherein the body comprises a three layer thermoplastic structure comprising:
an inner layer comprising an inner surface area of the body and defining the cavity;
an outer layer comprising an outer surface area of the body distal to the cavity and defining the first and second. pressure surfaces and the pair of opposing treatment surfaces; and
an intermediate layer disposed between the inner layer and the outer layer.

15. The therapeutic device of claim 14, wherein:
the inner layer comprises a poly(ethylene terephthalate) having a thickness of between about 0.09 mm to about 0.14 mm;
the intermediate layer comprises a nylon having a thickness of between about 0 to about 0.17 mm; and
the outer layer comprises a polyethylene having a thickness of between about 0.09 to about 0.14 mm.

16. The therapeutic device of claim 14, wherein tine inner layer is adapted to substantially impermeable to the composition disposed within the cavity.

17. The therapeutic device of claim 13, wherein the composition comprises a gel.

18. The therapeutic device of claim 17, wherein the gel comprises:
up to about 85% D-glucitol;
up to about 3% of a cross-linked polyacrylate copolymer; and
up to about 2% methylchloroisothiazolinone

19. The therapeutic device of claim 18, wherein the gel further comprises:
up to about 25% water; and
up to about 2% coloring.

20. A method of treating hemorrhoid disease, comprising:
cooling a body having a cavity containing a composition to a predetermined temperature, wherein the body is defined by:
a first end portion;
a second end portion;
a pair of opposing pressure surfaces extending between the first end portion and the second end portion comprising:
a first pressure surface having a first external surface area and
a second pressure surface having a second external surface area;
a pair of opposing treatment surfaces each extending between the first end portion and the second end portion and engaging the pair of opposing pressure surfaces;
positioning the body to engage an external skin surface at an external portion of an intergluteal space proximate to an anal verge, wherein:
the first external surface area of the first pressure surface engages an external skin surface portion of a first buttock such that substantially all of the first external surface area of the first pressure surface is in contact with the first buttock;
the second external surface area of the second pressure surface engages an external skin surface portion of an opposing second buttock such that substantially all of the second external surface area of the second pressure surface is in contact with the opposing second buttock;
the first and second pressure surfaces retain the body in position against the first buttock and the opposing second buttock; and
at least one of the pair of opposing treatment surfaces engages the external skin surface at the external portion of the intergluteal space proximate to the anal verge; and
allowing the body to remain in position for a predetermined amount of time.
